(19) United States
(12) Patent Application Publication
(10) Pub. No.: US 2022/0236249 A1
(43) Pub. Date: Jul. 28, 2022

(54) APPARATUS AND METHOD FOR SPUTUM CONDITIONING AND ANALYSIS

(71) Applicant: KONINKLIJKE PHILIPS N.V., EINDHOVEN (NL)

(72) Inventors: Kiran Hamilton J. Dellimore, Eindhoven (NL); Harold Johannes Antonius Brans, Eindhoven (NL); Marco Baragona, Eindhoven (NL); Samer Bou Jawde, Cambridge, MA (US)

(21) Appl. No.: 17/544,062

(22) Filed: Dec. 7, 2021

Related U.S. Application Data
(60) Provisional application No. 63/141,234, filed on Jan. 25, 2021.

(51) Int. Cl. G01N 33/487

(52) U.S. Cl. CPC G01N 33/487

(57) ABSTRACT

According to an aspect, there is provided an apparatus for sputum conditioning and analysis. The apparatus comprises: a microfluidic device configured to receive a sputum sample and to separate the sputum sample into a plurality of droplets; a biosensor configured to analyze each of a predetermined number of droplets of the plurality of droplets to acquire measurements of a characteristic of each droplet of the predetermined number of droplets; and a processor configured to analyze the acquired measurements to determine a characteristic of the sputum.

### CROSS-REFERENCE TO RELATED APPLICATIONS

This patent application claims the priority benefit under 35 U.S.C. § 119(e) of U.S. Provisional Application No. 63/141,234, filed on Jan. 25, 2021, the contents of which are herein incorporated by reference.

### FIELD OF THE INVENTION

The present invention relates generally to sputum conditioning and analysis for determination of sputum characteristics.

### BACKGROUND OF THE INVENTION

Many patients with chronic respiratory diseases, such as, for example, chronic obstructive pulmonary disease (COPD), cystic fibrosis (CF) and non CF-bronchiectasis, experience severe mucus build up in their airway system. Consequently, clearing the airways from mucus build up may become more difficult. This may lead to accumulation of bacterial load, which leads to exacerbations. Various pharmaceutical and non-pharmaceutical methods are typically employed to first loosen and/or thin the mucus prior to expulsion by coughing. Non-pharmaceutical loosening and/or thinning of mucus is usually achieved by manual (for example, chest percussion by a respiratory therapist) or semi-automated means (for example, high frequency chest wall oscillation therapy or Oscillating Positive Expiratory Pressure).

A key unmet need in non-pharmaceutical mucus loosening, thinning and clearance remains the optimization of the semi-automated therapy to meet patient-specific mucus removal needs in a domestic setting. This necessitates quantification of the amount of mucus build up (i.e., how much mucus needs to be removed), the distribution of mucus in the airway and the physical properties of the mucus, such as, for example, the mucus viscosity, stickiness, solid fraction, etc. Once obtained, this information may be used to personalize semi-automated mucus loosening, thinning and clearance therapy, by adapting the duration, frequency and/or device settings (for example, applied pressure, force, etc.).

Characterization and measurement of mucus physical properties is challenging due to a number of factors, including the complex and heterogeneous composition of mucus, limitations in collection methods, and laborious procedures for analysis of mucus. In domestic settings in particular, reliably and reproducibly obtaining suitable mucus samples (from the lower airways predominantly) via spontaneous sputum expectoration during coughing imposes further challenges because of saliva contamination and variations in the quantity of sputum produced. Moreover, heterogeneity and non-uniformity of the sputum samples can also make quantification of mucus properties difficult and imprecise. Without appropriate characterization and measurement of sputum and mucus, optimization and personalization of semi-automated mucus clearance therapy may not be possible.

It is therefore desirable to condition and analyze sputum (which may comprise mucus) to determine characteristics and properties of the sputum (and mucus). The determined characteristics and properties may then be used to control and personalize mucus loosening, thinning and clearance therapies and associated device settings, thereby enabling the improvement of the effectiveness of the therapies.

### SUMMARY OF THE INVENTION

According to an embodiment of a first aspect, there is provided an apparatus for sputum conditioning and analysis, the apparatus comprising: a microfluidic device configured to receive a sputum sample and to separate the sputum sample into a plurality of droplets; a biosensor configured to analyze each of a predetermined number of droplets of the plurality of droplets to acquire measurements of a characteristic of each droplet of the predetermined number of droplets; and a processor configured to analyze the acquired measurements to determine a characteristic of the sputum.

Thus, the sputum sample may be appropriately separated into droplets and measurements may be acquired from each of a number of droplets. Microfluidics allow for the separation and conditioning of the sputum into a plurality of droplets. The individual droplets may have consistent sizes allowing for measurements to be acquired from each droplet. From these measurements acquired from the droplets, a characteristic of the sputum may be determined, with measurement analytics applied to the droplet measurements to minimize the effects of inhomogeneity and saliva contamination on the quantification of the sputum qualities. The characteristic of the sputum may be indicative of physical properties of the sputum. Conditioning the sputum may be considered as preparing the sputum for analysis.

Embodiments of aspects may therefore address the problem of sputum sample contamination and inhomogeneity which makes the measurement of physical properties of mucus in a domestic setting unreliable and imprecise. In addition, embodiments of aspects may also address the problem of sample preconditioning which can be burdensome to the user or a time-consuming intermediate step before reliable measurements can be acquired.

Sputum is a heterogeneous material consisting of cells and mucus expelled from the lower airways of a user or patient via coughing. The sputum sample may be expectorated by a user and introduced to the microfluidic device. That is, the sputum sample may be received from a user, patient or individual. The sputum sample may be provided from a user or patient, such as, for example, an individual who suffers from a chronic respiratory disease and requires mucus loosening, thinning and clearance therapies. Variations in sputum collection may be minimized by following a fixed protocol. For example, sputum may be collected at a standard time during the day (for example, in the morning, after therapy, etc.), the user may be told to avoid eating or drinking for a period of time before providing the sputum sample, and the user may be instructed to rinse their mouth with water prior to providing the sputum sample. Such a protocol may minimize the largest differences in quantity and contaminations yet conditioning of the sputum is still required due to the heterogeneity and non-uniformity of the sputum, as discussed above.

The sputum may comprise mucus and the determined characteristic of the sputum may be indicative of the physical properties of the mucus. The sputum may be considered as a mixture of saliva, mucus and contaminants such as food etc., which makes it difficult to separate out the mucus from the sputum sample. However, a characteristic of the sputum determined by the apparatus may be indicative of a characteristic of the mucus. Thus, mucus properties may be considered to correspond to the sputum properties after analysis.

Invention embodiments are also applicable to a mucus sample (i.e. mucus separated from saliva, food, etc.), yet the difficulty in removing the mucus from the sputum makes sputum sample analysis more practicable. Sputum characteristics, though not fully indicative of mucus properties, highly depend on the properties of mucus. Thus, a characteristic of the sputum determined by the apparatus may be considered to correspond to a characteristic of mucus contained in the sputum.

The biosensor may also be referred to as a biosensing element or a biosensing device. The biosensor may comprise an optical sensor and/or an electrochemical sensor. An optical sensor may, for example, measure optical density and an electrochemical sensor may, for example, measure a biomarker such as mucin. The biosensor may be one of a plurality of biosensors arranged sequentially. Each biosensor may be configured to acquire measurements for one or more characteristics of the droplets. The characteristics may range and include, for example, physical properties to analytes.

The microfluidic device may be configured to transport the predetermined number of droplets to the biosensor. The processor may be configured to control the microfluidic device. The processor may be configured to control the biosensor.

The processor may be configured to output the determined sputum characteristic. The determined sputum characteristic may be output to one or more of: a display device of the apparatus; and a transmitter of the apparatus. That is, the apparatus may comprise a display device configured to display the determined sputum characteristic, and/or the apparatus may comprise a transmitter configured to transmit the determined sputum characteristic. The transmitter may be configured to transmit (output) the determined sputum characteristic to a networked device. That is, a device that is communicably connected to the apparatus. The device may, for example, be a user device (such as, for example) associated with the user. Additionally or alternatively, the device may be a therapy device for providing mucus loosening, thinning and clearance therapy to the user. The determined sputum characteristic may therefore be communicated to the user or transmitted to another device for presentation to the user.

The apparatus may comprise a memory configured to store the determined characteristic of the sputum. A number of determined characteristics of the sputum may be stored in the memory and may be stored in accordance with a time stamp of identifier of the sputum sample from which the characteristic was determined. The processor may be configured to analyze the stored sputum characteristics to determine differences between the characteristics and to determine trends over time. Each of the number of determined characteristics stored in the memory may be determined using samples of the same or comparable size and/or droplets of the same or comparable size. For example, the size of the samples and/or droplets may all be within a range of 10% either side of a target size, i.e. ±10% of a target liquid volume. Statistical analysis between samples may therefore be improved and less burdensome, since the statistics would be more comparable between samples.

Trends and differences between samples may be important in a domestic environment and the differences and trends in sputum properties may be used to help guide and optimize loosening and clearance therapy. That is, analysis of the sputum may capture changes occurring with mucus (and changes in the patient's condition) over time which may then be used to optimize therapies. A sample taking protocol may be carried out by the user prior to each sample analysis to minimize the differences between the samples and reduce the influence on the samples by external factors (such as, for example, food).

The processor may be configured to generate an alert in response to a difference between the determined characteristic and a preceding determined characteristic stored in the memory exceeding a predetermined threshold. That is, the processor may generate an alert if the variance between two determined characteristics measured at two adjacent time points is greater than a threshold level. Alternatively or additionally, the processor may be configured to generate an alert in response to a rate of change of a plurality of determined characteristics stored in the memory exceeding a predetermined threshold. That is, the processor may generate an alert if the determined characteristic varies over time to a degree that is greater than a threshold level. The processor may be configured to output the alert. Thus, in either or both cases, an alert may be generated and output to the user. For example, the user may be alerted to a large change in the sputum characteristic (such as, for example, viscosity) which may indicate a change in a medical condition. The user may therefore take appropriate action, such as, for example, adjust the therapy settings to account for the change or contact a healthcare professional. The alert may be a visual notification and/or an audio notification provided to the user, for example, via a user device connected to the apparatus or via a user interface (for example, a display) provided as part of the apparatus.

The processor may therefore be configured to monitor trends in the sputum characteristics and provide an alarm to the user/patient if there is a significant difference or deterioration of the sputum characteristic, such as, for example, if the viscosity of the sputum (and therefore the mucus) increases to a very high level in comparison to an expected level of viscosity. A large change in measurement data (increase or decrease) may result from a change in the patient's condition.

A large change in the determined characteristic may also indicate that the sample taking was not correctly performed. The processor may therefore generate an alert which instructs the user to provide another sample to the apparatus and the sputum conditioning and analysis may be performed on the new sputum sample. For example, a notification may be displayed to repeat the whole measurement. The alert/notification may also comprise a reminder of a protocol to be followed when providing a sputum sample. The processor may be configured to generate and output an alternative alert if the redetermined characteristic is consistent with the preceding characteristic, i.e. if the redetermined characteristic indicates that the sample taking was correctly performed. The alert may instruct the user to perform alternative actions, such as, for example, contacting a medical professional. Retaking the sample may provide confirmation that the measurement was correctly performed.

The microfluidic device may be a gradient device comprising an inlet, an upper plate and a lower plate. The sputum sample may be introduced to the microfluidic device via the inlet. The sputum sample may be introduced between the upper plate and the lower plate and separated into the plurality of droplets by a gradient of the upper plate and the lower plate.

The processor may be configured to control the gradient of the upper plate and the lower plate. Thus, the processor may control the separation and transportation of the sputum droplets. The gradient may be an electromechanical, chemical, topological or pressure gradient. The gradient device may therefore be used and controlled to control the separation of the sputum sample into droplets and the transportation of the droplets through the microfluidic device.

Each of the upper plate and the lower plate may comprise a plurality of electrowetting tiles. One or more of the electrowetting tiles may be coated with a dielectric layer. A dielectric coating may therefore be applied to the electrowetting tiles which may prevent molecules of the sputum from sticking to the tiles. This may therefore prevent contamination of the microfluidic device.

The microfluidic device may be an acoustical device comprising an inlet and a nebulizer. The sputum sample may be introduced to the microfluidic device via the inlet. The sputum sample may be separated into the plurality of droplets by the nebulizer. That is, the nebulizer may be used and controlled to separate the sputum into a plurality of droplets and to transport the droplets through the microfluidic device. The nebulizer may be considered as an acoustical element or an acoustical stimulus. It may be considered that the nebulizer vaporizes the sputum.

The acoustical device may comprise a sensing plate. The sensing plate may be coated with a dielectric layer. A dielectric coating may therefore be applied to the sensing plate which may prevent molecules of the sputum from sticking to the plate. This may therefore prevent contamination of the microfluidic device, which may alter measurements and cause errors.

The microfluidic device may be configured to receive a plurality of cleaning droplets. The microfluidic device may be configured to transport the plurality of cleaning droplets through the microfluidic device. Cleaning droplets may therefore be introduced to and transported through the microfluidic device to remove the sputum from the device and prevent contamination, which may alter measurements and cause errors. The apparatus may comprise a cleaning reservoir configured to store a carrier fluid and to introduce a plurality of cleaning droplets to the microfluidic device.

The processor may be configured to count the predetermined number of droplets. The processor may be configured to group the droplets in accordance with the acquired measurements. The processor may be configured to analyze the acquired measurements in accordance with the droplet count and the droplet grouping to determine the characteristic of the sputum. That is, the processor may count and group the droplets in accordance with the measurements so that a characteristic of the sputum may be identified. The processor may be configured to perform statistical analysis on the grouped droplet measurements to determine the sputum characteristic. Trends and common properties of the droplets may be identified by the grouping which enables the determination of the sputum characteristic. Grouping the droplets may also be considered as classifying the droplets. Filtering the droplets may comprise excluding droplets from the analysis.

The processor may count the number of droplets that have been analyzed by the biosensor. The processor may therefore obtain a count of the number of measurements acquired from the sputum sample. The number of droplets for statistical analysis may be predetermined or analysis of a minimum number of droplets may be required. Droplets may be excluded from analysis by the processor if they do not fit certain specification limits (i.e. outliers may be excluded from analysis). The number of droplets to be analyzed by the biosensor may be prescribed a priori and may be set by the processor and/or the user. This links to the sample size which may also specified beforehand.

The processor may be configured to filter the acquired measurements in accordance with a predetermined condition. The processor may be configured to analyze the acquired measurements in accordance with the filtered measurements to determine the characteristic of the sputum. That is, certain droplet measurements may be excluded from the analysis performed by the processor to determine the characteristic of the sputum. The excluded droplet measurements may correspond to outliers and the accuracy of the sputum characteristic determination may be improved by excluding droplets from the analysis.

The predetermined condition may correspond to a characteristic of the sputum. In other words, the condition on which it is determined to exclude droplet measurements may correspond to the characteristic to be determined. For example, if viscosity is the characteristic to be determined, then the predetermined condition may be a viscosity level such that, for example, measurements above a viscosity level are excluded. The filtering of droplet measurements may be performed in accordance with known properties of aspects of the sputum that are not desired in the analysis, such as, for example, food and/or saliva. Such exclusions may therefore result in the determined sputum characteristic more accurately reflecting a characteristic of mucus in the sputum. The mucus properties may therefore be determined by exclusion of the droplets that are contaminated, for example, with saliva and/or food.

The sputum may comprise mucus. The processor may be configured to determine a characteristic of the mucus in accordance with the characteristic of the sputum.

The apparatus may comprise a fluid reservoir. The fluid reservoir may be configured to store a carrier fluid. The fluid reservoir may be configured to introduce the carrier fluid to one or more of: the sputum sample; and each of the predetermined number of droplets. That is, the carrier fluid may be mixed with the sputum sample and/or the sputum droplets to provide a predetermined number of mixed droplets. The biosensor may be configured to perform the analysis on the mixed droplets. A known volume of the carrier fluid may be introduced to the sample and/or each droplet.

The analysis of the sputum may therefore be performed on sputum mixed with the carrier fluid, for which the properties are known. The homogeneity of the droplets may therefore be improved, which may improve the characteristic analysis of the sputum. The carrier fluid may also be referred to as a diluent, dilutant, thinner, and/or diluting agent. The carrier fluid may be saline solution. Mixing the sputum with a carrier fluid such as saline solution may lower the viscosity of the sample. The sputum sample and/or sputum droplets may also be mixed with a PBS solution, or other buffers that may be used to stabilize biological samples. If the volume of each constituent (the sputum and the carrier) are known, the properties of the carrier are known and the properties of the mixed sample (the sputum mixed with the carrier) are known, the processor may estimate the sputum property. That is, the properties of the sputum may be estimated if both the properties of the carrier and the mixed sample are known.

The apparatus may comprise a microfluidic peristaltic mixer. The microfluidic peristaltic mixer may be configured to mix the carrier fluid with the one or more of: the sputum sample; and each of the predetermined number of droplets. The microfluidic peristaltic mixer may therefore ensure that the sputum sample and/or sputum droplets are mixed with the carrier fluid prior to analysis.

The apparatus may comprise a waste reservoir. The waste reservoir may be configured to receive one or more droplets of the plurality of droplets. That is, the apparatus may comprise a waste reservoir which collects the sputum droplets when they are no longer required, for example, after analysis. The microfluidic device may be configured to transport the one or more droplets to the waste reservoir. The waste reservoir may reduce the burden on the user and prevent sputum build up in the microfluidic device.

The characteristic of each droplet of the predetermined number of droplets may be one or more of: a property of the droplet; and a biomarker of the droplet. That is, the characteristic of the droplet may be a property of the droplet and/or a biomarker of the droplet. The property of the droplet may comprise one or more of: wettability; optical density; electrical conductivity; and refractive index. The biomarker of the droplet may comprise one or more of: mucins; inorganic salts; proteins; and enzymes.

Contact angle may be used as a measure of wettability. That is, the wettability may be used to determine a contact angle. Refractive index is reflective of the sputum viscosity and so viscosity may be estimated from refractive index. Refractive index may be correlated to the viscosity but, in the case of sputum and mucus, it is unlikely to be linear. Inorganic salts may comprise Na+, K+, Cl—, etc.

According to an embodiment of a second aspect, there is provided a method for sputum analysis, the method comprising: receiving a sputum sample; separating the sputum sample into a plurality of droplets; analysing each of a predetermined number of droplets of the plurality of droplets to acquire measurements of a characteristic of each droplet of the predetermined number of droplets; and analysing the acquired measurements to determine a characteristic of the sputum.

According to an embodiment of a third aspect, there is provided a computer program which when executed carries out a method for sputum analysis, the method comprising: receiving a sputum sample; separating the sputum sample into a plurality of droplets; analysing each of a predetermined number of droplets of the plurality of droplets to acquire measurements of a characteristic of each droplet of the predetermined number of droplets; and analysing the acquired measurements to determine a characteristic of the sputum.

Features and sub-features of the method and computer program aspects may be applied to the apparatus aspects and vice versa.

According to an embodiment of fourth aspect of the invention there is provided a non-transitory computer-readable medium storing a computer program as described above.

An apparatus or computer program according to preferred embodiments of the present invention may comprise any combination of the method aspects. Methods or computer programs according to further embodiments may be described as computer-implemented in that they require processing and memory capability.

The apparatus according to preferred embodiments is described as configured or arranged to, or simply “to” carry out certain functions. This configuration or arrangement could be by use of hardware or middleware or any other suitable system. In preferred embodiments, the configuration or arrangement is by software.

Thus according to one aspect there is provided a program which, when loaded onto at least one computer configures the computer to become the apparatus according to any of the preceding apparatus definitions or any combination thereof.

According to an aspect there is provided a program which when loaded onto the at least one computer configures the at least one computer to carry out the method steps according to any of the preceding method definitions or any combination thereof.

In general, the computer may comprise the elements listed as being configured or arranged to provide the functions defined. For example, this computer may include memory, processing, and a network interface.

The invention may be implemented in digital electronic circuitry, or in computer hardware, firmware, software, or in combinations of them. The invention may be implemented as a computer program or computer program product, i.e., a computer program tangibly embodied in a non-transitory information carrier, e.g., in a machine-readable storage device, or in a propagated signal, for execution by, or to control the operation of, one or more hardware modules.

A computer program may be in the form of a stand-alone program, a computer program portion or more than one computer program and may be written in any form of programming language, including compiled or interpreted languages, and it may be deployed in any form, including as a stand-alone program or as a module, component, subroutine, or other unit suitable for use in a data processing environment. A computer program may be deployed to be executed on one module or on multiple modules at one site or distributed across multiple sites and interconnected by a communication network.

Method steps of the invention may be performed by one or more programmable processors executing a computer program to perform functions of the invention by operating on input data and generating output. Apparatus of the invention may be implemented as programmed hardware or as special purpose logic circuitry, including e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions coupled to one or more memory devices for storing instructions and data.

The invention is described in terms of particular embodiments. Other embodiments are within the scope of the following claims. For example, the steps of the invention may be performed in a different order and still achieve desirable results.

Elements of the invention have been described using the terms “memory”, “processor”, etc. The skilled person will appreciate that such terms and their equivalents may refer to parts of the system that are spatially separate but combine to serve the functions defined. Equally, the same physical parts of the system may provide two or more of the functions defined.

For example, separately defined means may be implemented using the same memory and/or processor as appropriate.

### BRIEF DESCRIPTION OF THE DRAWINGS

Exemplary embodiments will now be described, by way of example only, with reference to the following drawings, in which:

FIG. 1 is a block diagram of main apparatus components according to a general embodiment of an aspect of the invention;

FIG. 2 is a flowchart of a method according to a general embodiment of an aspect of the invention;

FIG. 3 is a diagram of an apparatus including a microfluidic device according to an embodiment of an aspect of the invention;

FIG. 4 is a diagram of an apparatus including a microfluidic device according to an embodiment of an aspect of the invention;

FIG. 5 is a graph showing classification of droplets according to an embodiment of an aspect of the invention;

FIG. 6A is a diagram showing mixing of sputum with a carrier fluid according to an embodiment of an aspect of the invention;

FIG. 6B is a diagram of sputum mixing according to an embodiment of an aspect of the invention;

FIG. 7 is a graph showing classification and filtering of droplets according to an embodiment of an aspect of the invention; and

FIG. 8 is a hardware diagram illustrating hardware that may be used to implement invention embodiments.

### DETAILED DESCRIPTION OF EMBODIMENTS

Embodiments of the present disclosure and the various features and advantageous details thereof are explained more fully with reference to the non-limiting examples that are described and/or illustrated in the drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the present disclosure. The examples used herein are intended merely to facilitate an understanding of ways in which the embodiments of the present may be practiced and to further enable those of skill in the art to practice the same. Accordingly, the examples herein should not be construed as limiting the scope of the embodiments of the present disclosure, which is defined solely by the appended claims and applicable law.

It is understood that the embodiments of the present disclosure are not limited to the particular methodology, protocols, devices, apparatus, materials, applications, etc., described herein, as these may vary. It is also to be understood that the terminology used herein is used for the purpose of describing particular embodiments only, and is not intended to be limiting in scope of the embodiments as claimed. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which the embodiments of the present disclosure belong. Preferred methods, devices, and materials are described, although any methods and materials similar or equivalent to those described herein may be used in the practice or testing of the embodiments.

Embodiments of aspects may provide an apparatus, method and computer program for sputum conditioning/preparation and analysis so as to determine a characteristic of the sputum. The characteristic of the sputum may be used to determine and optimize the application and settings of (non-pharmaceutical, semi-automated) mucus loosening, thinning and clearance therapies, such as, for example, those used in a domestic setting.

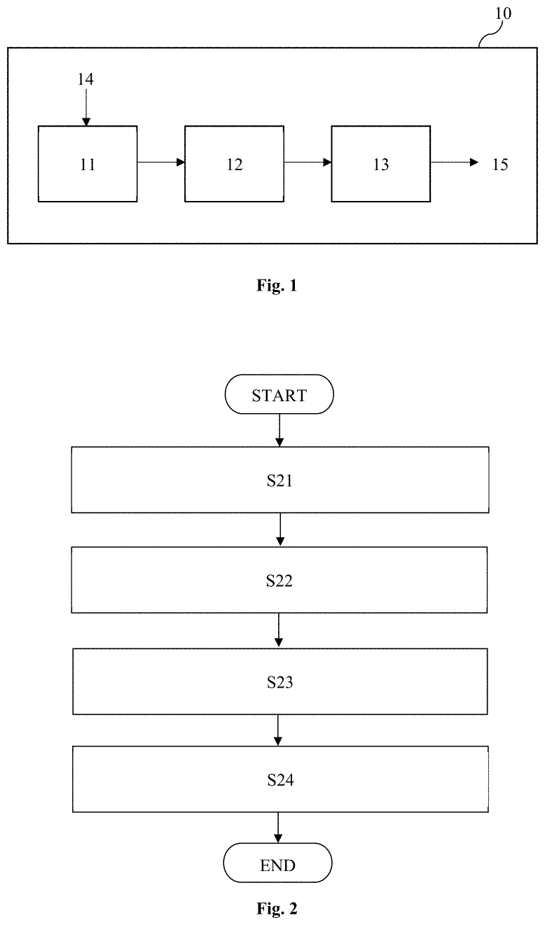

FIG. 1 shows a block diagram of information flow into main apparatus components in apparatus 10. The apparatus 10 comprises a microfluidic device 11, a biosensor 12 and a processor 13. A sputum sample 14 is received by the microfluidic device 11 and the microfluidic device 11 separates the sputum sample 14 into a plurality of droplets. The droplets are transported to the biosensor 12 and the biosensor 12 analyzes each droplet from a subset of the plurality of droplets to acquire measurements of a characteristic of each droplet of the subset of analyzed droplets. The processor 13 analyzes the acquired measurements and determines a characteristic of the sputum 15 from the analyzed measurements.

FIG. 2 shows a flow chart representing the method according to a general embodiment of an aspect of the invention. Firstly, in step S21, a sputum sample is received, and the sputum sample is separated into a plurality of droplets at step S22. Each of a predetermined number of droplets of the plurality of droplets are analyzed at step S23 to acquire measurements of a characteristic of each droplet of the predetermined number of droplets. Finally, at step S24, the acquired measurements are analyzed to determine a characteristic of the sputum.

Embodiments of aspects may therefore provide an apparatus, method and computer program to objectively and reliably assess sputum sample physical properties by minimizing the influence of contaminants and sputum inhomogeneity. In addition, embodiments of aspects may eliminate the need for burdensome and time-consuming sample preconditioning by utilizing the sputum sample ‘as is’ after expectoration from the user's respiratory system (lungs, throat, etc.).

As discussed above, the apparatus comprises a microfluidic device, a biosensor (biosensing element) and a processor (processing unit). The microfluidic device may comprise an inlet, an upper and lower plate with liquid transport. The liquid transport may be driven by an electromechanical (i.e., electrowetting), chemical, topological or pressure gradient, or by acoustical methods, such as, for example, surface acoustic waves (SAW) or an ultrasound, jet or vibrating nebulizer, or any combination thereof. A sputum sample may therefore be introduced to the microfluidic device (for example, from a user/patient), separated into droplets and then the droplets may be transported by the microfluidic device to the biosensing element.

The biosensing element may be composed of an optical or electrochemical sensor or detector, to measure physical properties and biomarkers, as well as count sputum droplets. The processing unit may control measurement acquisition, including: i) sputum droplet formation, ii) transport to the sensor or detector, and ii) analysis to determine the physical properties of the mucus. That is, the processing unit may control the microfluidic device and/or the biosensing element. By controlling the microfluidic device, the separation of the sputum sample into droplets may be controlled, as well as the transportation of the droplets to the biosensor. For example, the size and/or number of droplets may be controlled by the control of the microfluidic device through the processing unit.

The apparatus may also comprise a waste reservoir for disposal of droplets, for example, droplets that have been analyzed by the biosensor. Additionally or alternatively, the apparatus may comprise one or more fluid reservoirs to store a carrier fluid and/or a cleaning fluid. The carrier fluid may be added to the sputum droplets or the sputum, i.e. the carrier fluid may be mixed with the sputum droplets or the sputum sample. The cleaning fluid may be introduced to the apparatus to clean surfaces which have been in contact with a sputum sample.

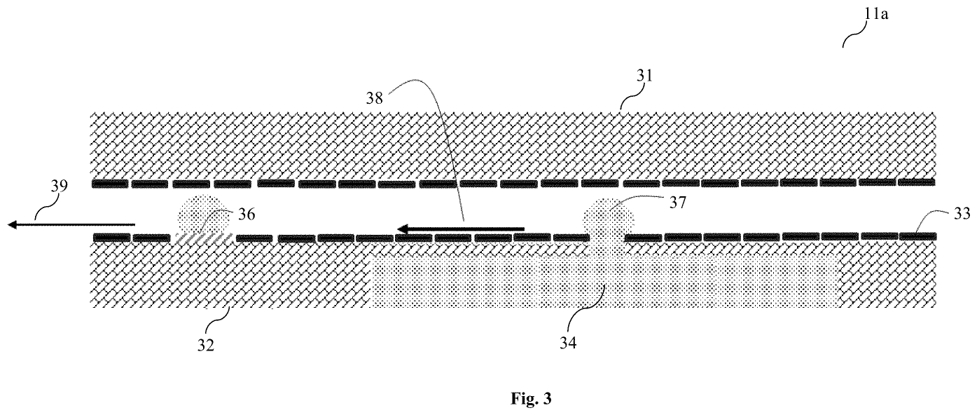

FIG. 3 shows a diagram of a microfluidic device according to an embodiment of an aspect of the invention. The microfluidic device of FIG. 3 may be considered as a gradient device. The device 11a comprises an upper plate 31, a lower plate 32 and a plurality of electrowetting (EW) tiles 33. The EW tiles 33 have a hydrophobic surface. The biosensor (sensing element) 36 is also provided in the microfluidic device 11a. A sputum sample 34 is introduced to the microfluidic device 11a and separated into droplets 37. The droplets 37 are transported through the microfluidic device 11a due to the gradient on the EW tiles 33, the direction of which is indicated by arrow 38. The droplet 37 is analyzed at the sensing element 36 to acquire a measurement of a characteristic of the droplet 37. After analysis at the sensing element 36, the droplet 37 is transported out of the microfluidic device 11a towards a waste reservoir (not shown), as indicated by the arrow 39. The inlet at which the sputum sample is introduced (for example, from a user) is also not shown.

The analysis of the sputum allows for the analysis of mucus present in the sputum sample. Embodiments of aspects may therefore reliably and accurately characterize mucus properties from a non-preconditioned, expectorated sputum sample by using a microfluidic device/system with a biosensing element (such as, for example, an electrochemical or optical sensor/detector) and a processing unit. The sputum sample or sub-sample may be introduced by the user into the microfluidic system via an inlet. The microfluidic device may comprise a gradient device which decomposes the sample into droplets by using an electromechanical gradient (i.e., electrowetting) applied along the upper and lower plates to ‘pinch off’ droplets of a prescribed volume. The droplet volume may, for example, be any whole or fractional number between (and including) 0.1 μl to 10 μl. The droplet volume is defined by the geometry of the upper and lower plates. The gradient device may also use a chemical or topological gradient for droplet formation and transport. However, these may provide less precise control and slower transport of the droplets when compared to the use of an electromechanical gradient (i.e. electrowetting).

The microfluidic device may enable a well-defined dislodgement of a droplet from the sputum sample using passive or active gradients (for example, electrowetting, chemical, topological or pressure) applied to the lower and/or upper plates of the microfluidic device. The volume of the droplet may be determined by the structure that detaches the droplet. Detachment must be achieved in a manner which ensures droplet disambiguation, i.e. unambiguous droplet definition.

Detachment of a droplet in the microfluidic device may be achieved using an interfacial tension method on the bottom plate of the microfluidic device. In this approach, detachment occurs on the moment that the hemispherical droplet reaches a certain diameter, on that size a passive gradient spanning the droplet diameter is sufficiently large to overcome the contact angle hysteresis of the droplet which is the phenomenon resisting movement. A passive gradient may be applied to the lower plate to achieve well-defined droplet detachment and the detachment occurs when the hemispherical droplet reaches a certain size (i.e. diameter). Once the droplet reaches this size, the gradient spanning the droplet diameter will be sufficiently large to overcome the contact angle hysteresis (i.e. the difference between the advancing and receding contact angles) of the droplet. It is this contact angle hysteresis which acts as a resistant force to the detachment by trying to retain the drop in its static position. After the droplet is detached then viscous drag also plays role in retarding droplet motion due to the driving force created by the surface energy gradient.

In the case of an active interfacial tension method, such as, for example electrowetting, applied to the lower plate, the detachment will take place when an electrowetting (i.e. electromechanical) wave is passing by and the droplet has a sufficient size to overlap at least partially two tiles of the electrowetting trajectory. EW allows better control of droplet size, once the height of microchannel is fixed

As discussed above with respect to FIG. 3, each droplet of the sputum sample (or each droplet of a subset of droplets) is transported sequentially to the biosensing element. The transport of the droplets can be actively controlled by applying an electromechanical gradient over a series of consecutive tiles of the upper and lower plates. The sputum droplets are then analyzed at the biosensing element to determine one or more physical properties. The biosensing element may use an optical sensor or an electrochemical sensor, which transduces the sputum droplet composition into an electrical signal, which is recorded using the processing unit. After a pre-determined number of the droplets derived from the sputum sample have been recorded, they are then analyzed by the processing unit. The number of droplets to be measured and recorded may be determined by the required measurement confidence level and/or the characteristic to be determined.

A broad range of physical properties and biomarkers may be measured. These may include: wettability (contact angle), optical density, electrical conductivity, refractive index (viscosity, which may, for example, be indirectly measured using the refractive index), mucins, inorganic salts (Na+, K+, Cl—, etc.), proteins and enzymes, etc. The physical properties and biomarkers may be collectively referred to as characteristics. The measured physical properties and/or biomarkers may be selected based on the disease and/or disease stage of the user that provided the sputum sample. That is, the physical properties and/or biomarkers to be measured may be selected in accordance with the user's condition, since, for example, certain physical properties and/or biomarkers provide a deeper insight into the patient status and/or may be more clinically useful for some diseases and conditions. The measured physical properties and biomarkers may also be selected based on the type of therapy to be provided to the user/patient.

Multiple sensing elements may be arranged sequentially, with one or more characteristics measured at each sensing element. For example, in cystic fibrosis (CF) a genetic mutation leads to defects in the cystic fibrosis transmembrane conductance regulator (CFTR) gene which encodes the CFTR channel protein which controls the flow of H2O and Cl— ions in and out of cells inside the lungs. When the CFTR protein is working correctly, ions freely flow in and out of the cells. However, when the CFTR protein is malfunctioning, these ions cannot flow out of the cell due to a blocked channel Thus, the mucus in CF patients is dry and sticky. The absence or lack of Cl— ions in a sputum sample may therefore be used to assess aspects such as mucolytic medication efficacy and adherence, as well disease progression. It may therefore be desirable to monitor such characteristics in a user/patient with CF. Accordingly, the characteristic to be determined may be determined based on a medical history of the user.

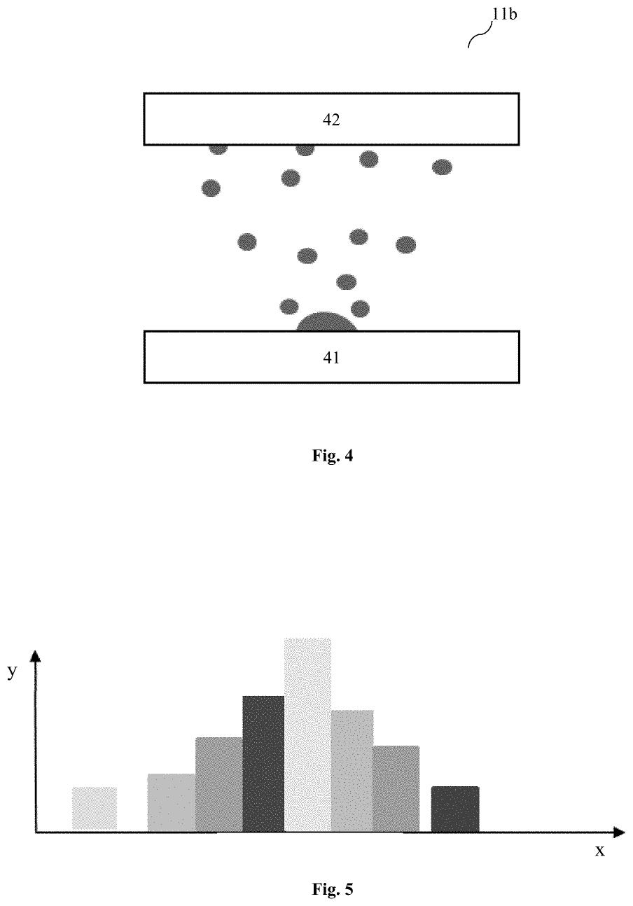

The microfluidic device shown in FIG. 3 is a gradient device. However, the microfluidic device may also be an acoustical device. FIG. 4 shows a diagram of a microfluidic device according to an embodiment of an aspect of the invention. The microfluidic device 11b of FIG. 4 may be considered as an acoustical device which forms and transports droplets of the sputum using acoustical methods, such as, for example, surface acoustic waves (SAW) or an ultrasound, jet or vibrating nebulizer.

The microfluidic device 11b of FIG. 4 comprises a nebulizer 41 and a sensing plate 42. The nebulizer 41 may be an ultrasound, jet or vibrating nebulizer or may provide SAW. The nebulizer 41 causes a jet or spray composed of a distribution of droplets in a given size range detectable by the biosensor(s). These droplets can then be collected on the sensing plate 42, and the sensing plate may comprise one or more biosensors to acquire the characteristic measurements of the droplets.

The collection of droplets may be made more effective by controlling the flow and transport of the spray. This can be accomplished by charging the aerosols with an induction charger, with such charging techniques known in the art. By giving the aerosols charge, the deposition of the droplets on the sensing plate may be controlled. For example, by coating the specific ‘unwanted’ areas with the same charge and coating other ‘wanted’ areas (such as, for example, the inlet of the microfluidic system) with an opposite charge. The droplets may be formed and transported in a less controlled manner using the acoustical device compared with the gradient device.

The processor (processing unit) may count the droplets and analyze the recorded droplet measurements. For example, the droplets can be classified according to discrete ranges of the mucus sample property or analyte of interest. For example, if the measured physical property is contact angle (i.e. wettability) on a hydrophobic or hydrophilic surface, the droplets may be counted and grouped according to contact angle (θ) ranges such as, for example θ<90°; 90°≤θ<100°; 100°≤θ<110°, 110°≤θ<120°; and θ>120°. In yet another example, the physical property measured may be optical density (OD), which may result in droplet OD ranges such as, for example OD<2; 3≤OD<4; 4≤OD<5; 5≤OD<6; and OD>6. Once the analysis on the individual droplets has been finalized, further statistical analysis may be performed to determine the mean, median and standard deviation of the mucus properties for the whole sample. That is, statistical analysis may be performed to determine trends and differences between samples which are indicative of the sputum (and mucus) characteristics. In the case of biomarkers, the droplets may be classified according to their concentration, count, absence or presence, or statistical distribution across droplets. This droplet analysis may permit a thorough, statistical characterization of sputum samples which is currently not possible in domestic settings.

FIG. 5 shows a graph of classification of droplets according to an embodiment of an aspect of the invention. The y-axis of FIG. 5 represents a droplet count and the x-axis represents a property or biomarker of the sputum/mucus, such as, for example, viscosity, optical density, etc. The graph of FIG. 5 therefore shows the output of the droplet characteristic analysis in which the droplets are counted and the measurements are classified. Statistical analysis of the classified measurements may then be performed.

In practice, a typical sputum sample volume of ˜10 ml may be assumed, of which a small fraction, such as, for example, between 1 μl and 100 μl, may undergo droplet analysis. For instance, a sub-sample volume of 1 μl, would yield ˜10,000 droplets, while 100 μl would yield 1 M droplets of the same size. In terms of analysis time, droplet samples may be transported to the sensing element and analyzed in milliseconds if an electromechanical gradient is applied. For 10,000 droplets, assuming a separation time between droplets of 10 ms to 100 ms, the total analysis time would be in the range of 100 s to 16.67 mins, which would be acceptable for a user in a domestic context. Alternatively, the sub-sample volume may be increased to, for example, between 1 ml and 5 ml to help reduce the impact of impurities on the measured physical properties. In that case, larger droplets may be formed on the order of, for example, between 10 μl and 50 μl respectively (to obtain around 100 droplets for analysis).

Taking a larger sample may further help reduce the effect of the impurities since the sample would be more representative. Due to the inhomogeneity of a sputum sample, a large sample volume may be favorable. After mixing with a saline solution, a fraction of this total volume may be used for droplet formation. A volume size of between 10 μl to 50 μl may be preferable. The sputum sample size may be determined to be representative of the sputum and the mucus in the sputum. As stated above, a larger sample size may be beneficial. The droplet size may be application dependent.

Using smaller droplets may allow for a better resolution and idea on the heterogeneity of the sample (if mixing has not occurred). Thus, if an estimate of bulk viscosity is required then the droplet size may be larger. Conversely, smaller droplets may be preferable if an understanding of the heterogeneity in the sputum is required.

Furthermore, it is important to emphasize that is possible for multiple different mucus properties and analytes to be measured simultaneously or consecutively. For example, the wettability and optical density may be measured at the same time or, in another scenario, optical density may be measured by an optical sensor followed by a biomarker such as mucin or Cl— content, measured by an electrochemical sensor. Thus, multiple biosensors may be provided and each biosensor may measure one or more characteristics of the sputum.

According to an embodiment of an aspect, the mucus property measurement accuracy may be enhanced by pre-mixing the sputum droplets with a known volume of carrier fluid such as, for example, saline solution, before droplet analysis. Mixing the sputum with a carrier fluid such as saline solution lowers the viscosity of the sample. The sputum sample and/or sputum droplets may also be mixed with a PBS solution, or other buffers that may be used to stabilize biological samples.

The carrier fluid may be stored in a fluid reservoir which is in fluidic contact with the microfluidic system. Addition of carrier fluid to the sputum droplets may increase the homogeneity of the droplets and thereby increase the consistency, precision and reliability of the sensor measurements. The carrier fluid may also be added in situations in which the sputum sub-sample or part of the sub-sample is too viscous or heterogeneous to support uniform droplet formation, such as, for example, if the sputum sub-sample is very heterogeneous with components which are, for instance, high in mucin or protein content. In these cases, it may also be advantageous to form larger droplets.

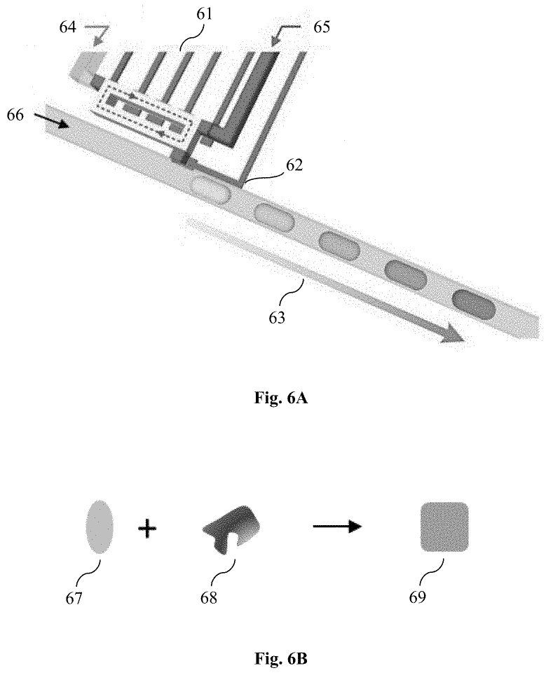

FIG. 6A shows a diagram of mixing sputum with a carrier fluid according to an embodiment of an aspect of the invention. In particular, FIG. 6A shows the function of a peristaltic microfluidic mixer 61 to pre-mix a sample with a carrier fluid (diluent). The diluent is introduced at 64 and the sputum sample is introduced at 65. The peristaltic microfluidic mixer 61 mixes the diluent and the sample and the mixed fluid is separated into droplets by a valve-assisted droplet generator 62. Oil is introduced at 66 and the arrow 63 indicates serial dilution of the sample. The oil may be used to aid droplet formation.

FIG. 6B shows a diagram of sputum mixing according to an embodiment of an aspect of the invention. In particular, FIG. 6B provides a representation of how the addition of carrier fluid to the sputum droplets may increase the homogeneity of the droplets. In FIG. 6B, the carrier fluid 67 is added to the sputum sample 68 to provide a homogenized sample 69.

The carrier fluid may be added to individual droplets and/or to the entire sputum sample prior to droplet formation. Mixing can be achieved by utilizing a microfluidic peristaltic mixer as discussed above with reference to FIG. 6A, with such mixers known in the art. Following the microfluidic analysis of the homogenized sample, and knowing the initial volume and properties (such as, for example, the density, viscosity, etc.) of the carrier fluid (for example, the saline solution), as well as the size of the mixed droplet, the mucus properties may be estimated. For example, the mucus viscosity could be estimated using Gambill's method, which is a technique for determining the viscosity of a two liquid mixture that is known in the art.

According to an embodiment of an aspect, droplet selection may be utilized. Droplet selection may improve the accuracy and reliability of the mucus property measurement. In this approach, certain droplet measurements are filtered and excluded from the analysis by the processor. For example, droplets with physical properties which fall in a range corresponding to sputum contaminants, such as, for example, saliva and food, may be selectively eliminated from the droplet statistical analysis. For example, it is known in the art that saliva is 99.5% water, while normal healthy mucus is about 98% water, and so droplets with a water content above 98% may be eliminated from the mucus characterization analysis. This range may also be adapted to account for the disease and disease stage of the patient. For instance, the water content of mucus from patients with cystic fibrosis is typically about 79%. Thus, droplets with water content above, for example, 79% may be selectively eliminated as they are likely to be contaminated. In yet another example, biochemical or rheological differences between mucus and sputum contaminants, such as food, may be exploited. For example, a droplet containing food particles will have biochemical components not expected in mucus such as, for example, carbohydrates (for example, glucose, fructose, starch, etc.) and lipids (for example, phospholipids, sulpholipids, etc.). Droplets with such components may therefore be excluded from the analysis performed by the processor to determine the characteristic of the sputum since these droplets are likely to contain food which would lead to inaccurate characterization. This embodiment may be applied to both a non-preconditioned and a homogenized sample, i.e. a sample that has been mixed with carrier fluid and one that has not.

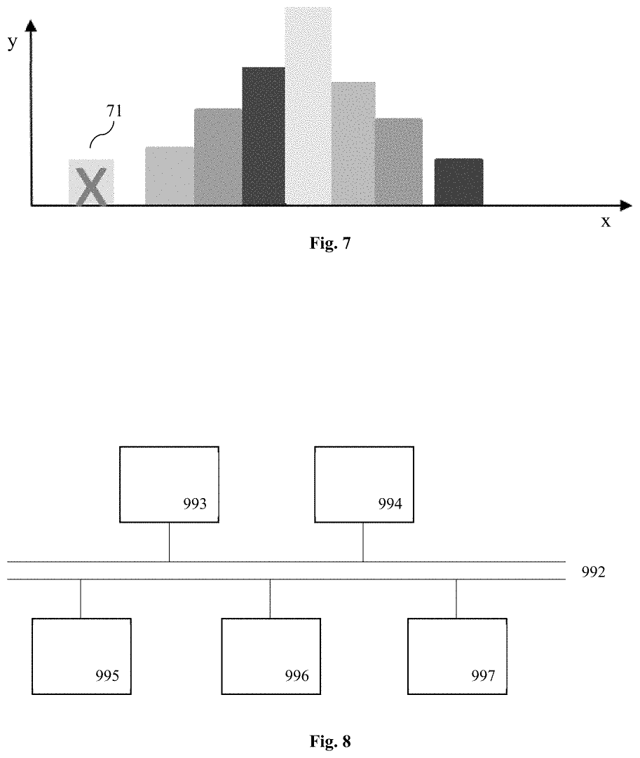

FIG. 7 shows a graph of classification and filtering of droplets according to an embodiment of an aspect of the invention. In the example shown in FIG. 7, the y-axis represents a droplet count and the x-axis represents a property or biomarker of the sputum/mucus. The range 71 is selectively eliminated from the droplet statistical analysis. As discussed above, the excluded range 71 may relate to mucus properties which fall in a range corresponding to sputum contaminants such as, for example, saliva and/or food.

According to an embodiment of an aspect, the apparatus may comprise means for cleaning the microfluidic device or preventing contamination. For example, the apparatus may comprise a fluid reservoir (cleaning reservoir) configured to store a cleaning fluid and to introduce the cleaning fluid to the microfluidic device. Storage and introduction of the cleaning fluid by the reservoir may be controlled by the processor and may reduce the burden on the user.

It is possible that the droplet formation and analysis system may become contaminated or fouled by previous sputum droplets. For example, proteins in the sputum droplets, which tend to stick to the electrowetting (EW) tiles or sensing plate surface, may contaminate the device and affect future analysis. This could make the hydrophobic surface of the electrowetting tiles hydrophilic thereby causing electrowetting to stop working. It could also influence the properties of subsequent droplets leading to less reliable characterization of the physical properties of the sputum sample. The cleaning means may therefore prevent these problems from occurring.

The cleaning means may comprise coating the electrowetting tiles or sensing plate surface with a dielectric layer which inhibits the sticking of molecules to the surface. Alternatively or additionally, the surfaces may be cleaned by periodically performing a cleaning step in which cleaning droplets are passed over the tiles. For example, the cleaning droplets may be introduced to and transported through the microfluidic device after a predetermined number of sputum droplets have been analyzed or in between samples, so as to clean the EW tiles. The cleaning droplets may be introduced by the user and/or from a fluid reservoir (cleaning reservoir). The cleaning droplets may be transported through the microfluidic device in the same way as the sputum droplets. The cleaning droplets may be composed of cleaning agents, including those especially designed for removing biologicals like proteins (such as, for example, Enzybrew 10 which is used in the beer brewing industry), thereby facilitating the removal of fouling substances from the system.

Embodiments of aspects may therefore provide an apparatus and method for conditioning and analyzing a sputum sample so as to determine a characteristic of the sputum. Embodiments of aspects may therefore enable more accurate and reliable quantification of mucus physical properties from a sputum sample by minimizing contamination effects and/or measurement of small sample volumes. The determined characteristic may be used in the determination and optimization of the application and settings of non-pharmaceutical, semi-automated mucus loosening, thinning and clearance therapies, such as those used in a domestic setting. The application of these therapies to an individual may therefore be improved and the effectiveness increased.

According to embodiments of aspects, microfluidic techniques are used, which may enhance the reliability and accuracy of mucus property measurement. In particular, problems related to sample conditioning and inhomogeneity may be addressed. According to an embodiment of an aspect, a microfluidic system is provided which separates sputum samples into (for example, ˜μl) droplets and actively or passively transports the droplets to a sensor (biosensor) which characterizes one or more physical properties of the sputum droplets (such as, for example, viscosity, stickiness and solid fraction). Droplet statistics may then be performed to obtain a reliable quantification of the characteristics (such as, for example, the physical properties) of the sputum sample, which may include capturing the degree of heterogeneity. According to an embodiment of another aspect, the sputum droplets may be premixed with a carrier fluid (for example, saline solution with known volume and physical properties). This may reduce the level of inhomogeneity in the sputum sample and enhance processing of the physical property measurements by applying droplet segregation.

Embodiments of aspects may, for example, be applied in domestic settings during COPD, CF and/or NM patient self-care/self-management to quantify the properties of expectorated mucus in order to guide therapy or disease management similar to guidance that is currently provided in hospital settings to help clinicians provide better respiratory healthcare. They may be used to support mucus clearance via various methods, such as, for example, OPEP, HFCWO, and/or manual chest percussion.

FIG. 8 is a block diagram of a computing device, such as a server incorporating resources suitable for sputum conditioning and analysis processing, which may embody the present invention, and which may be used to implement some or all of the steps of a method embodying the present invention, and perform some or all of the tasks of an apparatus of an embodiment. For example, the computing device of FIG. 8 may be used to implement all, or only some, of steps S21 to S24 of the method illustrated in FIG. 2, and to perform all, or only some, of the tasks of the apparatus shown in FIG. 1 to perform all, or only some, of the tasks of microfluidic device 11, biosensor 12 and/or processor 13. The computing device comprises a processor 993, and memory 994. Optionally, the computing device also includes a network interface 997 for communication with other computing devices, for example with other computing devices of invention embodiments.

For example, an embodiment may be composed of a network of such computing devices. Optionally, the computing device may also include one or more input mechanisms 996 such as a keyboard and mouse for the user to input any of, for example, user data or an image for analysis, and a display unit 995 such as one or more monitors. The display unit may show a representation of data stored by the computing device for instance, representations of the determined characteristic of the sputum. The display unit 995 may also display a cursor and dialogue boxes and screens enabling interaction between a user and the programs and data stored on the computing device. The input mechanisms 996 may enable a user to input data and instructions to the computing device. The components are connectable to one another via a bus 992.

The memory 994 may include a computer readable medium, which term may refer to a single medium or multiple media (e.g., a centralized or distributed database and/or associated caches and servers) configured to carry computer-executable instructions or have data structures stored thereon. Computer-executable instructions may include, for example, instructions and data accessible by and causing a general purpose computer, special purpose computer, or special purpose processing device (e.g., one or more processors) to perform one or more functions or operations. Thus, the term “computer-readable storage medium” may also include any medium that is capable of storing, encoding or carrying out a set of instructions for execution by the machine and that cause the machine to perform any one or more of the methods of the present disclosure. The term “computer-readable storage medium” may accordingly be taken to include, but not be limited to, solid-state memories, optical media and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

The processor 993 is configured to control the computing device and execute processing operations, for example executing code stored in the memory to implement the various different functions described here and in the claims. The memory 994 stores data being read and written by the processor 993, such as the inputs (such as, for example, the microfluidic device settings), interim results (such as, for example, the droplet measurements) and results of the processes referred to above (such as, for example, the characteristic of the sputum). As referred to herein, a processor may include one or more general-purpose processing devices such as a microprocessor, central processing unit, or the like. The processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also include one or more special-purpose processing devices such as an application specific integrated circuit (ASIC), a field programmable gate array (FPGA), a digital signal processor (DSP), network processor, or the like. In one or more embodiments, a processor is configured to execute instructions for performing the operations and steps discussed herein.

The display unit 995 may display a representation of data stored by the computing device and may also display a cursor and dialog boxes and screens enabling interaction between a user and the programs and data stored on the computing device. The input mechanisms 996 may enable a user to input data and instructions to the computing device. The display unit 995 and input mechanisms 996 may form the output 26.

The network interface (network I/F) 997 may be connected to a network, such as the Internet, and may be connectable to other such computing devices via the network. The network I/F 997 may control data input/output from/to other apparatus via the network. Other peripheral devices such as microphone, speakers, printer, power supply unit, fan, case, scanner, trackerball etc. may be included in the computing device.

Methods embodying the present invention may be carried out on a computing device such as that illustrated in FIG. 8. Such a computing device need not have every component illustrated in FIG. 8 and may be composed of a subset of those components. A method embodying the present invention may be carried out by a single computing device in communication with one or more data storage servers via a network. The computing device may be a data storage itself storing the input content before and after processing and thus for example, the dialogue and/or trained model.

A method embodying the present invention may be carried out by a plurality of computing devices operating in cooperation with one another. One or more of the plurality of computing devices may be a data storage server storing at least a portion of the data.

Other hardware arrangements, such as laptops, iPads and tablet PCs in general could alternatively be provided. The software for carrying out the method of invention embodiments as well as input content, and any other file required may be downloaded, for example over a network such as the internet, or using removable media. Any dialogue or trained model may be stored, written onto removable media or downloaded over a network.

The invention embodiments may be applied to any field in which effective and reliable analysis of sputum is desired. The invention embodiments may preferably be applied to the healthcare field, and particularly to the field of mucus loosening, thinning and clearance therapies in a user/patient.

Variations to the disclosed embodiments may be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored or distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

The above-described embodiments of the present invention may advantageously be used independently of any other of the embodiments or in any feasible combination with one or more others of the embodiments.

1. An apparatus for sputum conditioning and analysis, the apparatus comprising:
a microfluidic device configured to receive a sputum sample and to separate the sputum sample into a plurality of droplets;
a biosensor configured to analyze each of a predetermined number of droplets of the plurality of droplets to acquire measurements of a characteristic of each droplet of the predetermined number of droplets; and
a processor configured to analyze the acquired measurements to determine a characteristic of the sputum.

2. The apparatus of claim 1, wherein
the microfluidic device is a gradient device comprising an inlet, an upper plate and a lower plate;
the sputum sample is introduced to the microfluidic device via the inlet; and
the sputum sample is introduced between the upper plate and the lower plate and separated into the plurality of droplets by a gradient of the upper plate and the lower plate.

3. The apparatus of claim 2, wherein
each of the upper plate and the lower plate comprise a plurality of electrowetting tiles; and
one or more of the electrowetting tiles are coated with a dielectric layer.

4. The apparatus of claim 1, wherein
the microfluidic device is an acoustical device comprising an inlet and a nebulizer,
the sputum sample is introduced to the microfluidic device via the inlet; and
the sputum sample is separated into the plurality of droplets by the nebulizer.

5. The apparatus of claim 4, wherein
the acoustical device comprises a sensing plate; and
the sensing plate is coated with a dielectric layer.

6. The apparatus of claim 1, wherein the microfluidic device is configured to:
receive a plurality of cleaning droplets; and
transport the plurality of cleaning droplets through the microfluidic device.

7. The apparatus of claim 1, wherein the processor is configured to:
count the predetermined number of droplets;
group the droplets in accordance with the acquired measurements; and
analyze the acquired measurements in accordance with the droplet count and the droplet grouping to determine the characteristic of the sputum.

8. The apparatus of claim 1, wherein the processor is configured to:
filter the acquired measurements in accordance with a predetermined condition; and
analyze the acquired measurements in accordance with the filtered measurements to determine the characteristic of the sputum.

9. The apparatus of claim 1, wherein
the sputum comprises mucus; and
the processor is configured to determine a characteristic of the mucus in accordance with the characteristic of the sputum.

10. The apparatus of claim 1, comprising a fluid reservoir configured to store a carrier fluid and to introduce the carrier fluid to one or more of: the sputum sample; and each of the predetermined number of droplets.

11. The apparatus of claim 10, comprising a microfluidic peristaltic mixer configured to mix the carrier fluid with the one or more of: the sputum sample; and each of the predetermined number of droplets.

12. The apparatus of claim 1, comprising a waste reservoir configured to receive one or more droplets of the plurality of droplets.

13. The apparatus of claim 1, wherein the characteristic of each droplet of the predetermined number of droplets is one or more of: a property of the droplet; and a biomarker of the droplet.

14. A method for sputum analysis, the method comprising:
receiving a sputum sample;
separating the sputum sample into a plurality of droplets;
analysing each of a predetermined number of droplets of the plurality of droplets to acquire measurements of a characteristic of each droplet of the predetermined number of droplets; and
analysing the acquired measurements to determine a characteristic of the sputum.

15. A computer program which when executed carries out a method for sputum analysis, the method comprising:
receiving a sputum sample;
separating the sputum sample into a plurality of droplets;
analysing each of a predetermined number of droplets of the plurality of droplets to acquire measurements of a characteristic of each droplet of the predetermined number of droplets; and
analysing the acquired measurements to determine a characteristic of the sputum.
